(19) United States
(12) Patent Application Publication
(10) Pub. No.: US 2005/0154391 A1
(43) Pub. Date: Jul. 14, 2005

(54) Bone anchor assemblies

(76) Inventors: Thomas Doherty, Bellingham, MA (US); Mark Hall, Bridgewater, MA (US); David Selvitelli, Suffield, CT (US); Danielle Sheeran, Norfolk, MA (US); Thomas Runco, Canton, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/025,875

(22) Filed: Dec. 29, 2004

Related U.S. Application Data
(60) Provisional application No. 60/533,404, filed on Dec. 30, 2003.

(51) Int. Cl. A61B 17/56

(52) U.S. Cl. 606/61

(57) ABSTRACT

A bone anchor assembly may include a bone anchor having a distal shaft configured to engage bone and a proximal member. The proximal member may have a first section and a second section coupled to at least a portion of the bone anchor. The second section may be movably connected to the first section to facilitate relative rotation of the first section and the second section.

### REFERENCE TO RELATED APPLICATIONS

This application claims priority to U.S. Provisional Patent Application No. 60/533,404, filed Dec. 30, 2003, which is incorporated herein by reference.

### BACKGROUND

Spinal fixation systems may be used in orthopedic surgery to align and/or fix a desired relationship between adjacent vertebrae. Such systems typically include a spinal fixation element, such as a relatively rigid fixation rod or plate, that is coupled to adjacent vertebrae by attaching the element to various anchoring devices, such as hooks, bolts, wires, or screws. The spinal fixation element can have a predetermined contour that has been designed according to the properties of the target implantation site, and once installed, the spinal fixation element holds the vertebrae in a desired spatial relationship, either until desired healing or spinal fusion has taken place, or for some longer period of time.

Spinal fixation elements can be anchored to specific portions of the vertebra. Since each vertebra varies in shape and size, a variety of anchoring devices have been developed to facilitate engagement of a particular portion of the bone. Pedicle screw assemblies, for example, have a shape and size that is configured to engage pedicle bone. Such screws typically include a threaded shank that is adapted to be threaded into a vertebra, and a head portion having a spinal fixation element receiving element, which, in spinal rod applications, is usually in the form of a U-shaped slot formed in the head for receiving the rod. A set-screw, plug, cap or similar type of closure mechanism, may be used to lock the rod into the rod-receiving portion of the pedicle screw. In use, the shank portion of each screw may be threaded into a vertebra, and once properly positioned, a fixation rod may be seated through the rod-receiving portion of each screw and the rod is locked in place by tightening a cap or similar type of closure mechanism to securely interconnect each screw and the fixation rod. Other anchoring devices also include hooks and other types of bone screws.

In certain procedures, it may be difficult to position bone anchors on adjacent vertebrae because the close proximity of the adjacent vertebrae can result in interference between the bone anchors. In cervical vertebrae, for example, it is frequently necessary to pivot the bone anchors out of alignment with one another to avoid such interference.

### SUMMARY

Disclosed herein are bone anchor assemblies and methods of engaging a bone anchor assembly to bone that facilitate engagement of the bone anchor assembly to a bone, such as a vertebra. Also disclosed herein are methods of manufacturing a bone anchor assembly.

In one exemplary embodiment, a bone anchor assembly may comprise a bone anchor having a distal shaft configured to engage bone and a proximal member. In the exemplary embodiment, the proximal member may have a first section and a second section coupled to at least a portion of the bone anchor. The second section may be movably connected to the first section to facilitate relative motion of the first section and the second section.

An exemplary method of engaging a bone anchor assembly to a bone of a patient may comprise delivering a bone anchor assembly to proximate the bone. The bone anchor may comprise a bone anchor having a distal shaft configured to engage bone and a proximal member. The proximal member, in the exemplary embodiment, may have a first section and a second section coupled to at least a portion of the bone anchor. In the exemplary embodiment, the second section may be movably connected to the first section. The exemplary method may comprise engaging the shaft of the bone anchor to the bone and moving the first section relative to the second section.

### BRIEF DESCRIPTION OF THE FIGURES

These and other features and advantages of the bone anchor assemblies and methods disclosed herein will be more fully understood by reference to the following detailed description in conjunction with the attached drawings in which like reference numerals refer to like elements through the different views. The drawings illustrate principles of the instruments disclosed herein and, although not to scale, show relative dimensions.

FIG. 1 is a side elevational view of an exemplary bone anchor assembly;

FIG. 2 is a side elevational view of the bone anchor assembly of FIG. 1, illustrating the bone anchor positioned at multiple angular locations;

FIG. 3 is an exploded assembly view of the bone anchor assembly of FIG. 1, illustrating the components of the bone anchor assembly;

FIG. 4 is a side elevational view of the bone anchor of the bone anchor assembly of FIG. 1;

FIG. 5 is a side elevational view in cross section of the bone anchor of the bone anchor assembly of FIG. 1 taken along lines A-A of FIG. 4;

FIG. 6 is a perspective view of the first section of the receiving member of the bone anchor assembly of FIG. 1;

FIG. 7 is a top view of the first section of the receiving member of the bone anchor assembly of FIG. 1;

FIG. 8 is a side elevational view in cross section of the first section of the receiving member of the bone anchor assembly of FIG. 1 taken along the line B-B of FIG. 7;

FIG. 9 is a perspective view of the second section of the receiving member of the bone anchor assembly of FIG. 1;

FIG. 10 is a top view of the second section of the receiving member of the bone anchor assembly of FIG. 1;

FIG. 11 is a side elevational view in cross section of the second section of the receiving member of the bone anchor assembly of FIG. 1 taken along the line B-B of FIG. 10;

FIG. 12 is a side elevational view in cross section of a closure mechanism of the bone anchor assembly of FIG. 1;

FIG. 13 is a perspective view of a compression member of the bone anchor assembly of FIG. 1;

FIG. 14 is a side elevational view in cross section of the compression member of FIG. 13;

FIGS. 15A and 15B are perspective views of an exemplary bone anchor assembly;

FIG. 16 is a side elevational view of the bone anchor assembly of FIGS. 15A and 15B;

FIG. 17 is a side elevational view in cross section of the bone anchor assembly of FIGS. 15A and 15B;

FIG. 18 is an exploded assembly view of the components of the bone anchor assembly of FIGS. 15A and 15B;

FIG. 19 is a side elevational view in cross section of the components of the bone anchor assembly of FIG. 15A and 15B;

FIG. 20 is a perspective view of the first section of the receiving member of the bone anchor assembly of FIGS. 15A and 15B;

FIG. 21 is a side elevation view in partial cross section of the first section of the receiving member of the bone anchor assembly of FIGS. 15A and 15B;

FIG. 22 is a side elevation view in partial cross section of the distal end of the first section of the receiving member of the bone anchor assembly of FIGS. 15A and 15B;

FIG. 23 is a front view of the first section of the receiving member of the bone anchor assembly of FIGS. 15A and 15B;

FIG. 24 is a side elevational view of the first section of a receiving member of the bone anchor assembly of FIGS. 15A and 15B;

FIGS. 25A and 25B are perspective views of the second section of the receiving member of the bone anchor assembly of FIGS. 15A and 15B;

FIG. 26 is a side elevational view in cross section of the second section of the receiving member of the bone anchor assembly of FIGS. 15A and 15B; and

FIG. 27 is a side elevational view in cross section of the components of the bone anchor assembly of FIG. 15A and 15B, illustrating the relative dimensions of the components of the bone anchor assembly.

### DETAILED DESCRIPTION

Certain exemplary embodiments will now be described to provide an overall understanding of the principles of the structure, function, manufacture, and use of the bone anchor assemblies disclosed herein. One or more examples of these embodiments are illustrated in the accompanying drawings. Those of ordinary skill in the art will understand that the bone anchor assemblies specifically described herein and illustrated in the accompanying drawings are non-limiting exemplary embodiments and that the scope of the present invention is defined solely be the claims. The features illustrated or described in connection with one exemplary embodiment may be combined with the features of other embodiments. Such modifications and variations are intended to be included within the scope of the present invention.

The articles “a” and “an” are used herein to refer to one or to more than one (i.e. to at least one) of the grammatical object of the article. By way of example, “an element” means one element or more than one element.

The term “distal” as used herein with respect to any component or structure will generally refer to a position or orientation that is proximate, relatively, to the bone surface to which a bone anchor is to be applied. Conversely, the term “proximal” as used herein with respect to any component or structure will generally refer to a position or orientation that is distant, relatively, to the bone surface to which a bone anchor is to be applied.

The terms “comprise,” “include,” and “have,” and the derivatives thereof, are used herein interchangeably as comprehensive, open-ended terms. For example, use of “comprising,” “including,” or “having” means that whatever element is comprised, had, or included, is not the only element encompassed by the subject of the clause that contains the verb.

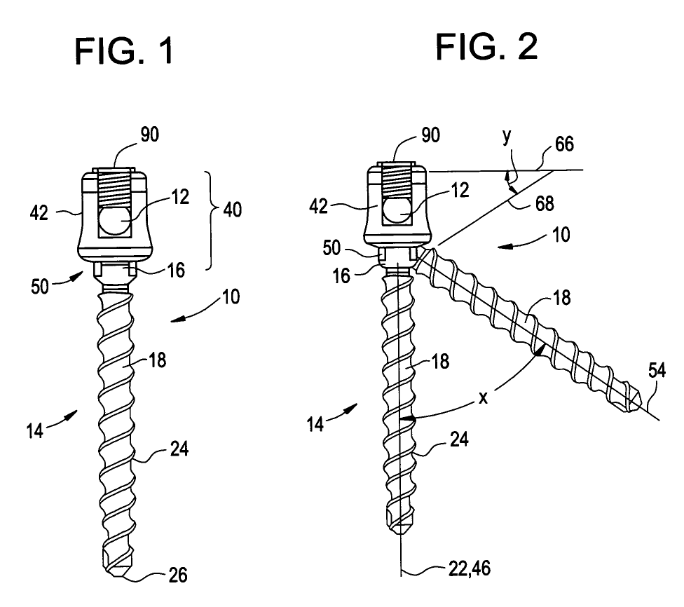

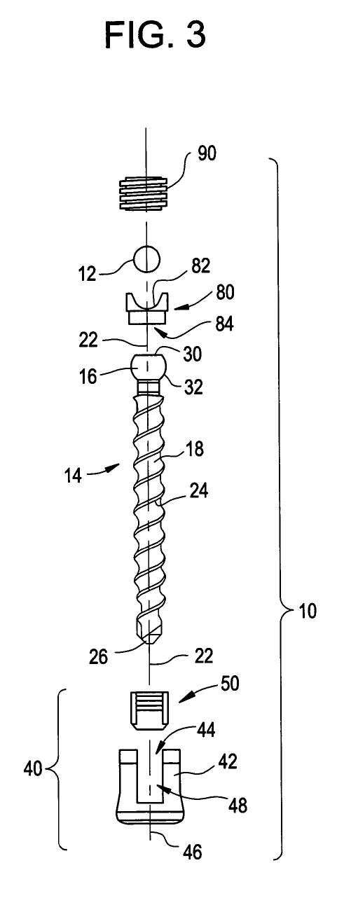

FIGS. 1-3 illustrate an exemplary embodiment of a bone anchor assembly 10 coupled to an exemplary spinal fixation element, a spinal rod 12. The exemplary bone anchor assembly 10 may be employed to engage one or more spinal fixation elements to bone. For example, bone anchor assembly 10 may be employed to fix a spinal plate, rod, and/or cable to a vertebra of the spine. Although the exemplary bone anchor assembly 10 described below is designed primarily for use in spinal applications, one skilled in the art will appreciate that the structure, features, and principles of the exemplary bone anchor assembly 10, as well as the other exemplary embodiments described below, may be employed to couple any type of orthopedic implant to any type of bone or tissue. Non-limiting examples of applications of the bone fixation anchor assembly 10 described herein include long bone fracture fixation/stabilization, small bone stabilization, lumbar spine as well as thoracic stabilization/fusion, cervical spine compression/fixation, and skull fracture/reconstruction plating.

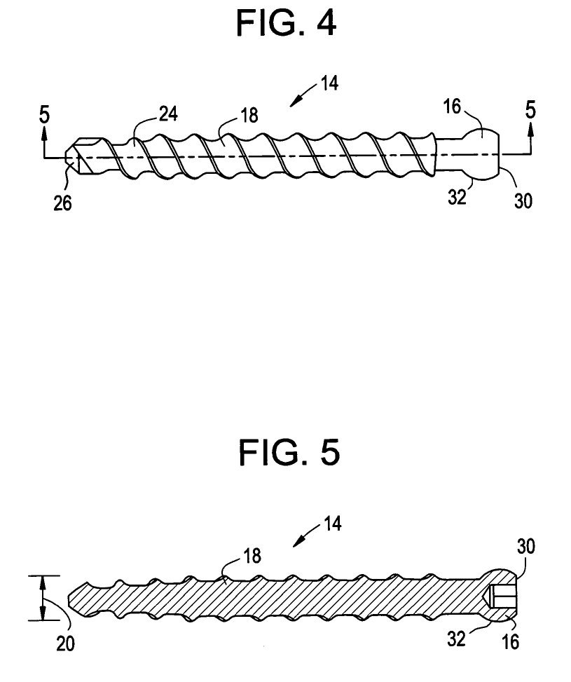

The illustrated exemplary bone anchor 10 may include a bone anchor 14 having a proximal head 16 and a distal shaft 18 configured to engage bone, as illustrated in FIGS. 1-5. The distal shaft 18 of the bone anchor 14 has a shaft diameter 20 and a longitudinal axis 22. The distal shaft 18 may include one or more bone engagement mechanisms to facilitate gripping engagement of the bone anchor 14 to bone. In the illustrated exemplary embodiment, for example, the distal shaft 18 includes an external thread 24. The external thread 24 may extend along at least a portion of the shaft 18. For example, in the illustrated exemplary embodiment, the external thread 24 extends from the distal tip 26 of the shaft 18 to proximate the head 16 of the bone anchor 14. One skilled in the art will appreciate that bone engagement mechanisms other than external thread 24 may be employed, including, for example, one or more annular ridges, multiple threads, dual lead threads, variable pitched threads, and/or any other conventional bone engagement mechanism. In the illustrated exemplary embodiment, the shaft diameter 20 of shaft 18 may be defined by the major diameter of external thread 24.

The proximal head 16 of the exemplary bone anchor 14 may be configured to facilitate adjustment of the bone anchor 14 relative to the receiving member 40 of the bone anchor assembly 10, as described below. For example, the head 16 may be generally spherical in shape to permit pivoting of the bone anchor 14 relative to the receiving member 40. In illustrated exemplary embodiment, for example, the head 16 may be in the shape of a truncated sphere having a generally planar proximal surface 30 and a generally hemispherically shaped distal surface 32. The head 16 of the bone anchor may have surface texturing, knurling, and/or ridges. The head 16 may also be in the shape of a sphere with more than one diameter. The centers of each spherical diameter may or may not be concentric.

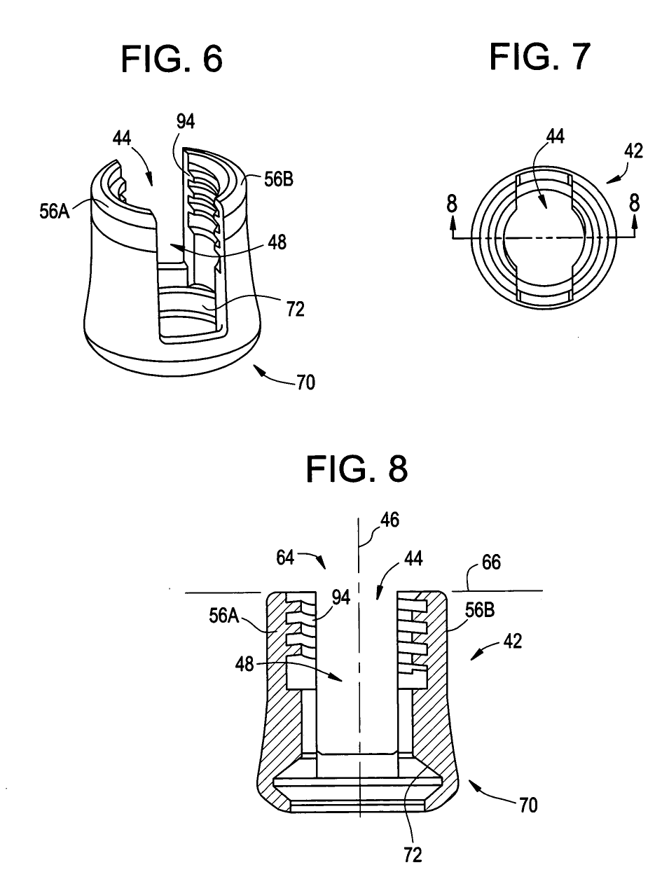

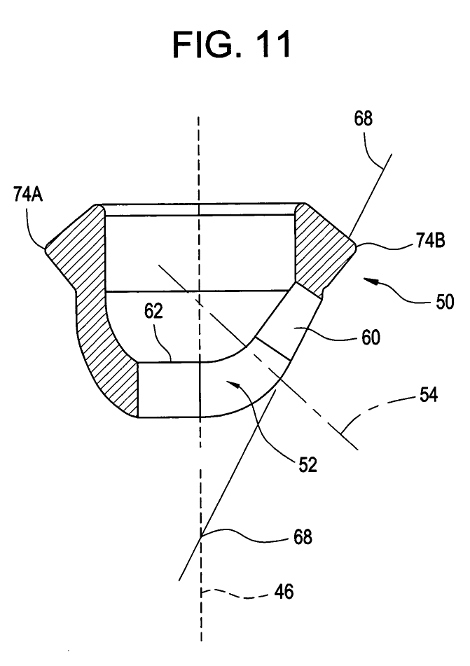

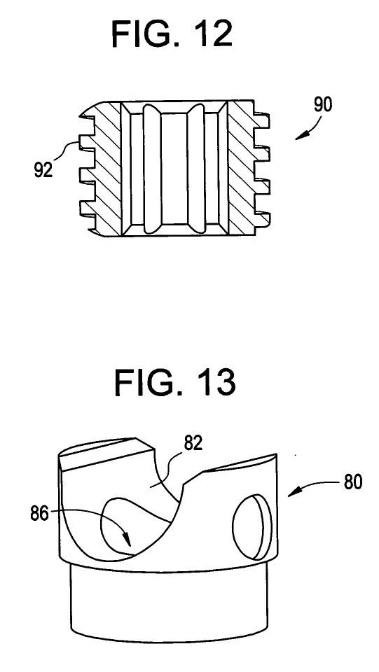

Referring to FIGS. 1-3 and 6-11, the receiving member 40 of the exemplary bone anchor assembly 10 includes a first section 42 having a first bore 44 defining a first bore axis 46, a recess 48 in communication with the first bore 44, and a second section 50 having a second bore 52. In the exemplary embodiment, the second bore 52 defines a second bore axis 54 that intersects the first bore axis 46, as discussed in more detail below. The first section 42 may be positioned at the proximal end of the receiving member 40 and the second section 50 may be positioned at the distal end of the receiving member 40, as in the illustrated exemplary embodiment.

The receiving member 40, in certain exemplary embodiments, may be configured to receive a spinal fixation element and couple the spinal fixation element to the bone anchor assembly. In the exemplary embodiment, for example, the recess 48 is provided in the first section 42 of the receiving member 40 and the recess 48 may be sized and shaped to receive a spinal rod 12, as illustrated in FIGS. 1-3. For example, the first section 42 of receiving member 40 has a generally U-shaped cross-section defined by two legs 56A and 56B separated by recess 48. Each leg 56A, 56B is free at the proximal end of the first section 42. The exemplary spinal rod 12 may be seated within the recess 48 by aligning the spinal rod 12 and the recess 48, advancing the spinal rod 12 through the first bore 44 into the recess 48. The configuration of recess 48 of the receiving member 40 may be varied to accommodate the type, size and shape of spinal fixation employed. In alternative exemplary embodiments, the exemplary spinal rod 14, or other spinal fixation element, may be coupled to the bone anchor assembly by alternative coupling mechanisms, in place of recess 48, including, for example, by an offset coupling mechanism, such as a band clamp, a sacral extender, or a lateral off-set connector

The receiving member 40 may couple a spinal fixation element to a bone anchor. In the exemplary embodiment, the second bore 52 of the second section 50 may have a first opening 60 through which at least a portion of a bone anchor, such as exemplary bone anchor 14 described above, may extend. For example, the shaft 18 of the exemplary bone anchor 14 may extend through the first opening 60, as illustrated in FIGS. 1-2. The first opening 60 may be sized and shaped to engage the head 16 of the exemplary bone anchor 14. For example, the first opening 60 may define a seat 62 for engaging the head 16 of the exemplary bone anchor 14 that allows the bone anchor 14 to pivot relative to the receiving member 40. In some exemplary embodiments, the seat 62 may be generally spherical in shape to permit pivoting of the bone anchor 14 relative to the receiving member. In the illustrated exemplary embodiment, the seat 62 may be generally hemispherical in shape and may have a curvature analogous to the distal surface 32 of the head 16 of the exemplary bone anchor 14. In other exemplary embodiments, the seat 62 may be tapered or may have any other shape that allows adjustment of the head of the bone anchor relative to the receiving member. In the exemplary embodiment, the bone anchor assembly 10 is a polyaxial bone anchor assembly as the bone anchor 14 may be pivoted to one or more angles relative to the receiving member 40. In particular, the bone anchor 14 may be adjusted such that the longitudinal axis 22 of the bone anchor 14 is at angle of 0° to 90° relative to the second bore axis 54. In other exemplary embodiments, the seat 62 may be provided by a separate component that fits within the receiving member, such as a snap ring.

One skilled in the art will appreciate that the bone anchor assemblies disclosed herein are not limited to the exemplary bone screw 14. In alternative exemplary embodiments, other bone anchors may be employed, including, for example, a monoaxial bone screw in which the bone screw is fixed relative to the receiving member, or a polyaxial or monoaxial hook or bolt.

The second bore axis 54 may be oriented at an angle to the first bore axis 46 to provided a preferred angle of orientation to the bone anchor. For example, the second bore axis 54 can be oriented at an angle X of approximately 0° to approximately 90° relative to the first bore axis 46, as illustrated in FIG. 2. In bone anchor assemblies designed for use in the spine, the second bore axis 54 may be oriented at an angle X of approximately 15° to approximately 70° relative to the first bore axis 46. For bone anchor assemblies used in the lower cervical, thoracic, and lumbar regions of the spine, the second bore axis 54 may be oriented at an angle X of approximately 20° relative to the first bore axis 46. For bone anchor assemblies used in the upper cervical, e.g., C1, C2, and the sacro-iliac regions of the spine, the second bore axis 54 may be oriented at an angle X of approximately 55° relative to the first bore axis 46.

In other exemplary embodiments, the second bore axis 54 may be coaxial to the first bore axis 46, i.e., the second bore axis 54 can be oriented at an angle X of approximately 0° relative to the first bore axis 46.

In the illustrated exemplary embodiment, the first bore 44 has a proximal opening 64 defining a first plane 66 and a portion of the first opening 60 defines a second plane 68. The first plane 66 may intersect the second plane 68 in the exemplary embodiment such that the second plane 68 is oriented at the angle Y relative to the first plane 66. In the exemplary embodiment, the angle Y may be approximately equal to the angle X. In other exemplary embodiments, the angle Y may be distinct from the angle X.

The second section 50 of the receiving member 40 may be rotatably connected to the first section 42 to facilitate relative rotation of the first section 42 and the second section 50. The second section 42 may seat internally within the first section 42, as in the illustrated exemplary embodiment, may externally connect to the first section 42, as in the bone anchor assembly 200 described below, and/or may connect in any other manner that allows the second section 50 to rotate relative to the first section 42.

In the illustrated exemplary embodiment, the distal end 70 of the first section 42 includes an annular groove 72 that is configured to receive one or more annular ridges 74 provided on the second section 50. For example, a pair of opposed ridges 74A and 74B may be provided proximate the proximal end of the second section 50. Any number of ridges may be provided, including, for example, a single annular ridge. When assembled, the ridge(s) 74 seat within the annular groove 72 and may rotate within the groove. The second section 50 may be rotatable 360° about the first bore axis 46 of the first section 42 by, for example, allowing the ridge(s) 74 to rotate through the entire extent of the groove 72. In certain exemplary embodiments, the second section 50 may be rotatable less than 360° about the first bore axis 46 of the first section 42 by, for example, providing one or more stops within the annular groove 72 or by varying the configuration of the groove and/or the ridge(s).

The bone anchor assembly 10 may optionally include a compression member 80 positionable within the receiving member 40 between the spinal fixation element and the bone anchor. The compression member 80 may be positioned within the first bore 44 and the recess 48 between the spinal rod 12 and the head 16 of the exemplary bone anchor 14. In the exemplary embodiment, the compression member 80 may have a proximal first surface 82 for engaging the spinal fixation element and an opposing distal second surface 84 for engaging the head 16 of the bone anchor 14.

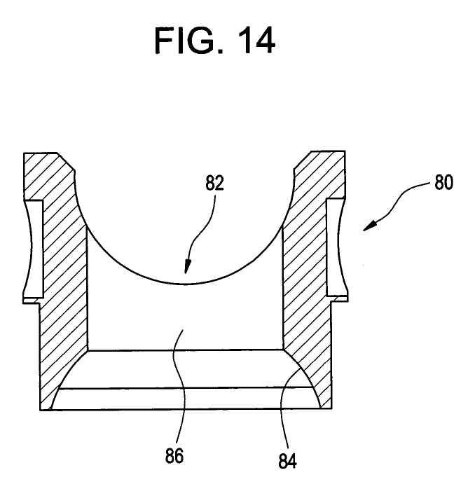

Referring to FIGS. 13 and 14, the exemplary embodiment of the compression member 80 may be generally disc-shaped having a circular cross-section or other cross section preferably analogous to the cross-section of the first bore 44 of the receiving member 40. The first surface 82 of the compression member 80 may be configured to seat the spinal fixation element. In the exemplary embodiment, the first surface 82 has a generally arcuate cross-section having a curvature that may approximate the curvature of the exemplary spinal rod 14. The second surface 84 may be configured to engage the head of the bone anchor. For example, the second surface 84 may have a generally spherical shape or a tapered shape to engage the head of the bone anchor. In the exemplary embodiment, the second surface 84 may have be hemispherical in shape and may have a curvature approximating the curvature of the head 16 of the bone anchor 14. A bore 86 may extend between the first surface 82 and the second surface 84 through an instrument may be advanced to the bone anchor 14 once the bone anchor assembly 10 is assembled.

The exemplary bone anchor assembly 10 may include a closure mechanism 90 that secures the spinal fixation element to the bone anchor assembly. Referring to FIGS. 1-3, the closure mechanism 90 secures the exemplary spinal rod 12 within the recess 48 of the receiving member 40. The closure mechanism 90 may engage the first section 42 of the receiving member 40 or, in other exemplary embodiments, may engage other portion(s) of the receiving member 40. The exemplary closure mechanism 90 is an internal closure mechanism that is positionable within the first bore 44 and engages an inner surface of the proximal end of the first section 42 of the receiving member 40. For example, the closure mechanism 90 may have external threads 92 that engage internal threads 94 provided on the first section 42 of the receiving member 40. Distal advancement of the closure mechanism 90 into engagement of the spinal rod 12, secures the spinal rod 12 within the recess 48 of the receiving member 40. In embodiments employing a compression member 80, such as exemplary bone anchor 10, distal advancement of the closure mechanism 90 into engagement with the spinal rod 12 seats the spinal rod 12 in the compression member 80. Distal advancement of the spinal rod 12 may also fix the bone anchor 14 relative to the receiving member 40 by engagement of the spinal rod 12 against the head 16 of the bone anchor 14 or by engagement of the compression member 80 against the head 16 of the bone anchor, as in the case of the illustrated exemplary embodiment. Advancement of the closure mechanism 90 may also lock the second section 50 to the first section 50. For example, in the illustrated exemplary embodiment, the head 16 of bone anchor 14 engages the second section 50 causing the ridges 74 to bear against the groove 72 and inhibit rotation of the ridges 74 within the groove 72.

One skilled in the art will appreciate that other types of closure mechanisms may be employed. For example, an external closure mechanism, such as an externally threaded cap, positionable about the first section 42 of the receiving member 40 may be employed. In other exemplary embodiments, the closure mechanism may comprise an external and an internal closure mechanism, a non-threaded twist-in cap, and/or any other conventional closure mechanism.

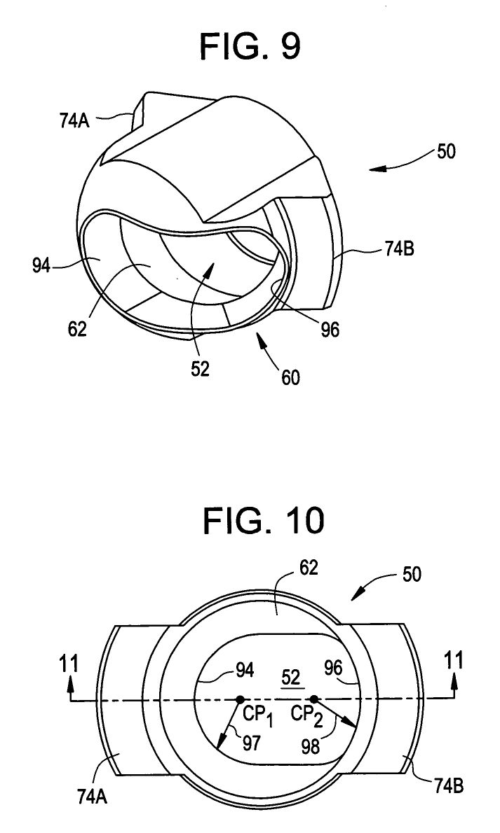

In the exemplary embodiment, the first opening 60 of the second section 50 of the receiving member 40 is configured to allow a portion of a bone anchor, such as the shaft 18 of the exemplary bone anchor 14, to be inserted therethrough during assembly of the bone anchor assembly 10. For example, the first opening 60 may be generally oblong in shape, as in the illustrated exemplary embodiment, and may be intersected by the first bore axis 46 and the second bore axis 54 when the first and second sections are assembled, as illustrated in FIGS. 9-11. In the exemplary embodiment, the first opening 60 may have a first arcuate end 94 spaced apart a distance from a second arcuate end 96. The distance between the first arcuate end 94 and the second arcuate end 96 may be selected such that the first bore axis 46 and the second bore axis 54 intersect the first opening 60. The first arcuate end 94 may have a center CP₁ that is proximate the first bore axis 46 and the second arcuate end may have a center CP₂ that is proximate the second bore axis 54. In certain exemplary embodiments, such as the illustrated exemplary embodiment, the first arcuate end 94 may have a center CP₁ that is intersected by the first bore axis 46 and the second arcuate end 96 may have a center CP₂ that is intersected by the second bore axis 54.

The first arcuate end 94 may have a first radius of curvature 97 distinct from the second radius of curvature 98 of the second arcuate end 96. For example, the first radius of curvature 97 may be greater or less than the second radius of curvature 98. The first radius of curvature 97 may be greater than the shaft diameter of the bone anchor to facilitate insertion of the bone anchor to the receiving member 40 during assembly. The first bore may include internal threads for engagement with threads provided on the shaft of the bone anchor to facilitate passage of the shaft through the first opening 60. The threads may extend to the first arcuate end 94, allowing the first arcuate end 94 to have a radius of curvature less than the shaft diameter of the bone anchor.

In other exemplary embodiments, the first arcuate end 94 may have a radius of curvature 97 approximately equal to the radius of curvature 98 of the second arcuate end 96, as in the case of the illustrated exemplary embodiment. In such embodiments, the first opening 60 may be generally elliptical in cross-section.

The components of the bone anchor assembly may be manufactured from any biocompatible material, including, for example, metals and metal alloys such as titanium and stainless steel, polymers, and/or ceramics. The components may be manufactured of the same or different materials. In one exemplary method of manufacturing, the bone anchor, the first section of the receiving member, and the second section of the receiving member are separately constructed and assembled prior to implantation. In one exemplary method of manufacturing, the second section 50 may be inserted through the first bore 44 and advanced distally to seat the ridge(s) 74 into the groove 72 of the first section 42. The recess 48 may acts as a keyway allowing the ridges 74A,B of the second section 50 to be advanced distally through the first bore of the second section 42. Once the ridges 74A,B are advanced to the groove 72, the second section 50 may be rotated to seat the ridges 74 in the groove 72.

A bone anchor, such as exemplary bone anchor 14, may be inserted into the receiving member 40 through the first bore 44. During insertion, the longitudinal axis of the bone anchor may be aligned with the first bore axis 46. At least a portion of the bone anchor, e.g., the shaft of the bone anchor, may be advanced through the first opening 60 of the second bore 52. During advancement, the longitudinal axis of the bone anchor may remain aligned with the first bore axis 46. The head of the bone anchor may be seated against seat 62 of the first opening 60 such that the shaft 18 of the bone anchor 14 extends through the first opening 60. The compression member 80 may be positioned through the first bore 44 into engagement with the head of the bone anchor before, or after, implantation of the bone anchor assembly.

In other exemplary embodiments, the bone anchor 14 may be inserted into the first opening 60 of the second section 50 prior to assembly of the second section 50 to the first section 42.

The bone anchor assembly 10 may be implanted by any conventional procedure. In one exemplary method of engaging the bone anchor assembly to a vertebra of the spine, the bone anchor assembly may be delivered to proximate the vertebra through an open incision or, in a minimally invasive procedure, though a percutaneous pathway between a minimally invasive skin incision and the vertebra. The second section 52, and the bone anchor connected thereto, may be rotated relative to the first section 42 to the desired orientation. A tool, such as bone anchor driver, may be inserted through the first bore 44 to engage the head of the bone anchor and may be employed to secure the bone anchor to the vertebra by, for example, rotating the proximal end of the tool. The tool can drive the bone anchor into a pre-drilled hole in the vertebra or, in the case of self-drilling bone screws for example, the tool can rotate the bone anchor and create a hole in bone as the bone anchor is advanced.

Depending on the procedure, a spinal fixation element may be coupled to the bone anchor assembly. Once the bone anchor engages the bone, the first section 42 may be rotated relative to the second section 50, and, thus, the bone anchor, facilitating alignment of the recess 48 with the spinal fixation element. The spinal fixation element may be coupled to the bone anchor assembly before, during, or after the bone anchor assembly engages the bone. A closure mechanism may be used to secure the fixation element to the bone anchor assembly.

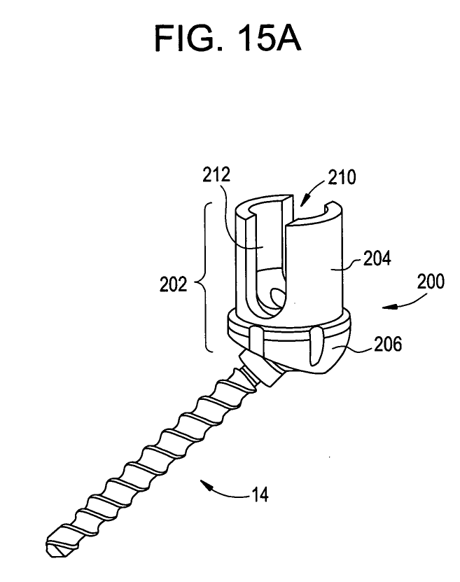

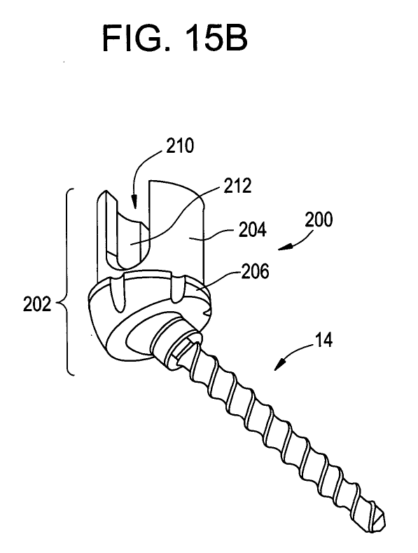

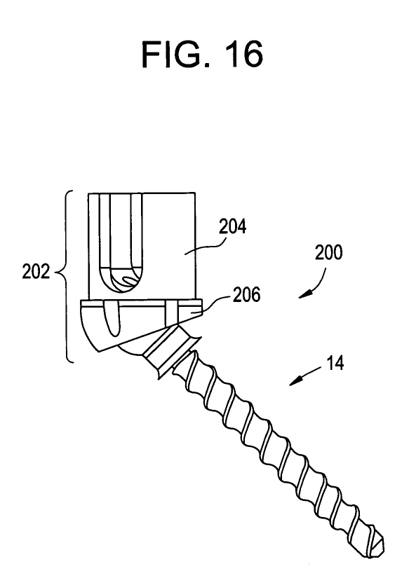

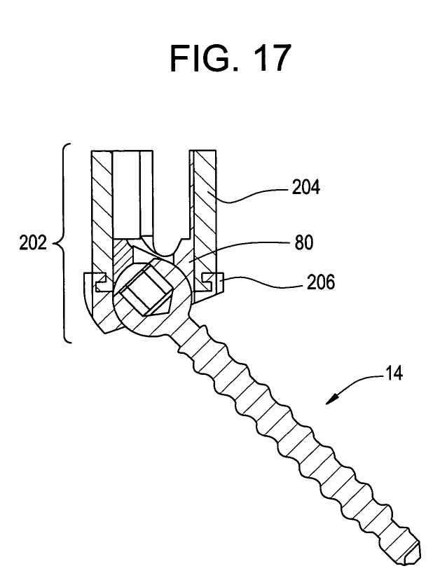

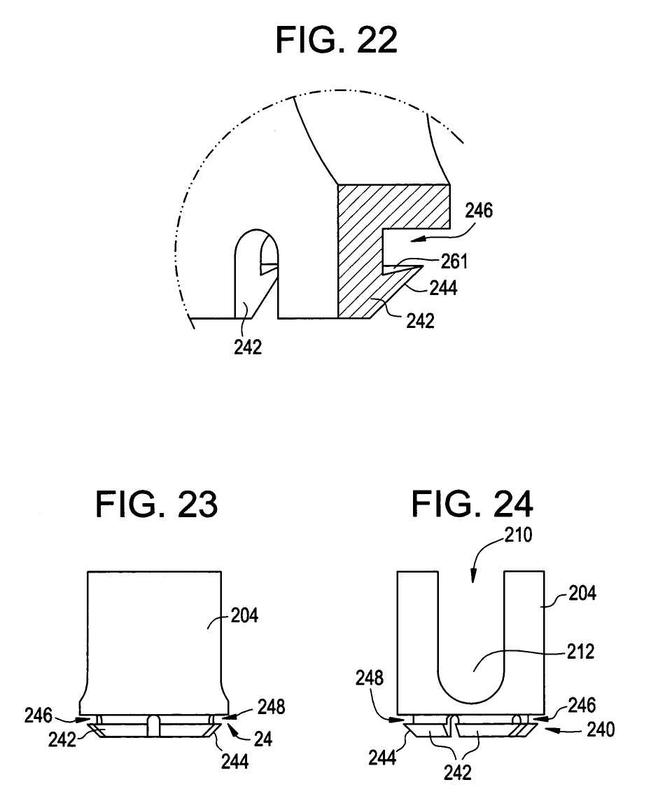

FIGS. 15A-26 illustrate an exemplary embodiment of a bone anchor assembly 200 including a proximal member 202 having a first section 204 and a second section 206 that may be rotatably connected to the first section 204 to facilitate relative rotation of the first section 204 and the second section 206 about the first bore axis 208 of the first section 204. The receiving member 202 may generally be analogous in construction to the receiving member 40 described above, except that the second section 206 may be externally rotatably connected to the first section 204, as described below.

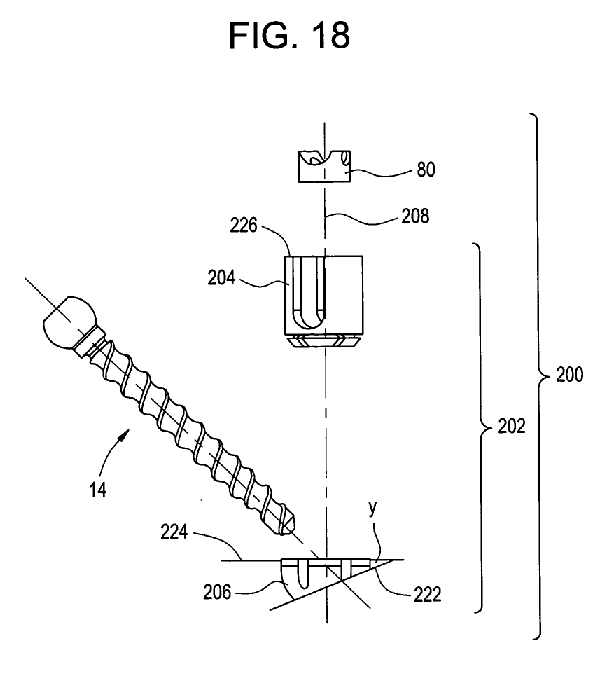

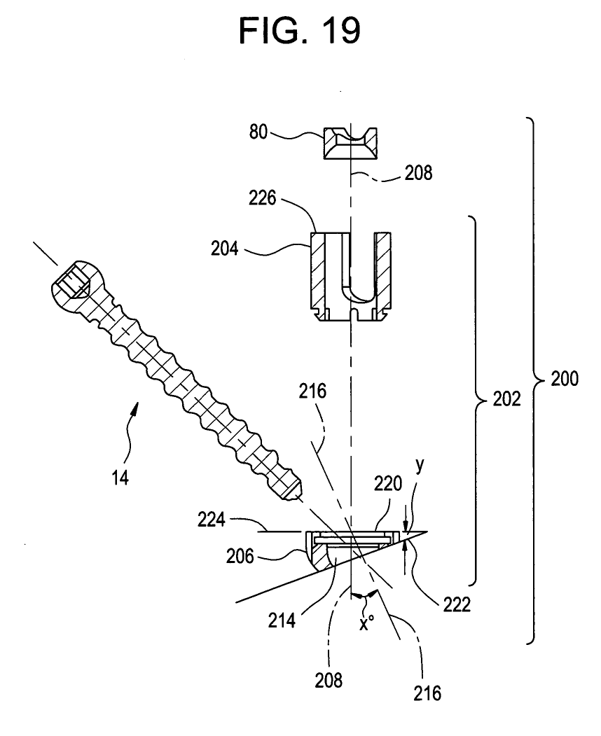

In the illustrated exemplary embodiment, the first section 204 may be configured in a manner analogous to the first section 42 of the bone anchor assembly 10 described above. For example, the first section 204 may include first bore 210 that communicates with a recess 212 for receiving a fixation element, such as a spinal rod. The second section 206 may be configured in a manner generally analogous to the second section 50 of the bone anchor assembly 10 described above. For example, the second section 206 may include a second bore 214 having a second bore axis 216, and a first opening 218 spaced apart from a second opening 220. The first opening 218 defines a first plane 222 and the second opening 220 defines a second plane 224 that intersects the first plane 222 at an angle Y, as illustrated in FIGS. 18 and 19. For example, the second plane 224 can be oriented at an angle Y of approximately 0° to approximately 90° relative to the first plane 224. The second plane 224 may be oriented approximately parallel to a plane defined by the proximal surface 226 of first section 204, as in the case of the illustrated exemplary embodiment, or may be oriented at an angle with respect to a plane defined by the proximal surface 224. The second bore axis 216 may be oriented at an angle X to the first bore axis 208. For example, the second bore axis 216 can be oriented at an angle X of approximately 0° to approximately 90° relative to the first bore axis 208. The angle X and the angle Y may be approximately equal, as in the case of the illustrated exemplary embodiment, or may be distinct. In bone anchor assemblies designed for use in the spine, the second bore axis 216 may be oriented at an angle X of approximately 15° to approximately 70° relative to the first bore axis 208. For bone anchor assemblies used in the lower cervical, thoracic, and lumbar regions of the spine, the second bore axis 216 may be oriented at an angle X of approximately 20° relative to the first bore axis 208. For bone anchor assemblies used in the upper cervical, e.g., C1, C2, and the sacro-iliac regions of the spine, the second bore axis 216 may be oriented at an angle X of approximately 55° relative to the first bore axis 208.

In other exemplary embodiments, the second bore axis 216 may be coaxial to the first bore axis 208, i.e., the second bore axis 216 can be oriented at an angle X of approximately 0° relative to the first bore axis 208.

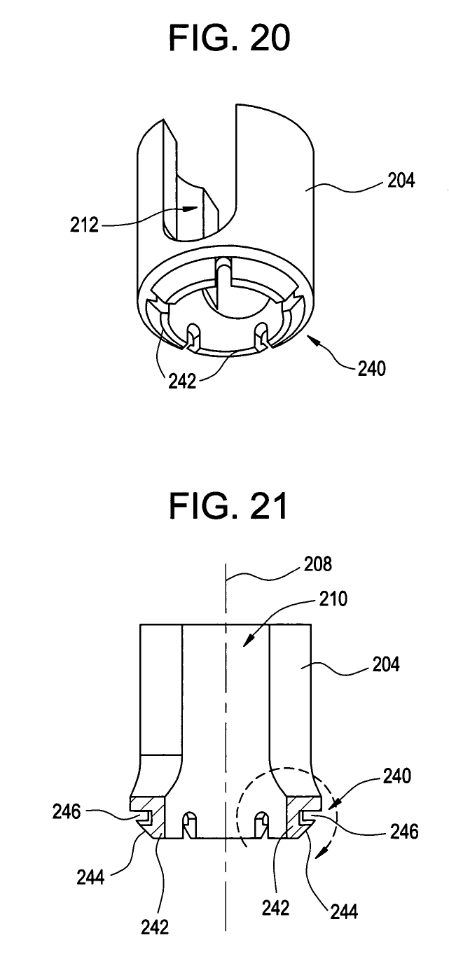

Referring to FIGS. 20-24, the first section 204 has a distal end 240 configured to rotatably engage the second section 206. For example, the distal end 240 may include one or more flexible, resilient fingers 242 that extend distally from the distal end 240. In the illustrated exemplary embodiment, for example, the distal end 240 includes five arcuately shaped fingers 242 spaced symmetrically about the circumference of the distal end 240. The fingers 242 may be radially inwardly flexible to allow a portion of the second section 206 to slide thereover and snap into place. Each finger 242 may have an angled distal surface 244 that facilitates advancement of the second section 206 over the fingers 242. Each finger 242 may include an arcuate groove 246. Collectively, the arcuate grooves may define a generally annular groove 248 into which an annular lip 250 provided on the second section 206 may be seated.

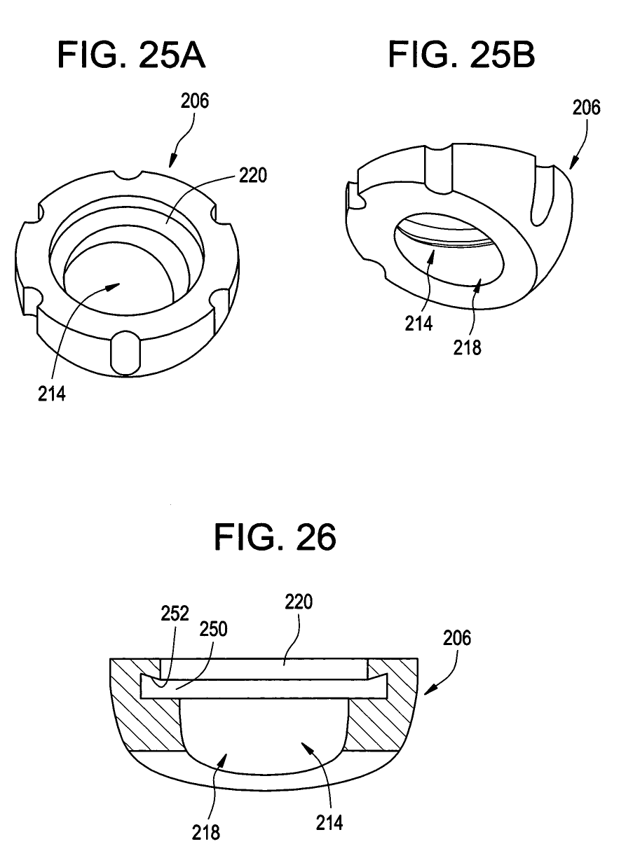

Referring to FIGS. 25A-26, the second section 206 may include an annular lip 250 that is configured to seat within the annular groove 248 and rotate within the annular groove 248. The annular lip 250 may be a continuous structure, as in the illustrated embodiment, or may be a plurality of spaced-apart arcuate components.

The second section 206 may be rotatable 360° about the first bore axis 208 of the second section 206. As the bone anchor 14 may be coupled to the second section 206, the bone anchor 14 may be rotated with the second section 206 about the first bore axis 208. One or more stops may be provided on the first and/or second section 204, 206 to limit the extent of second section 206 to less than 360°.

An optional compression member 80 may be positioned between the spinal fixation element and the head of the bone anchor, as in the exemplary bone anchor 10 described above. The compression member 80, when positioned, may inhibit radial flexing of the fingers 242, which inhibits separation of the second section 206 from the first section 204.

A closure mechanism 90 may be provided to secure a spinal fixation element to the bone anchor assembly 100 and to lock the second section 206 to the first section 204. For example, distal advancement of the closure mechanism 90 in the illustrated exemplary embodiment causes a bearing surface 252 provided on the annular lip 250 to engage a bearing surface 261 on each of the fingers 242, causing the second section 206 to lock to the first section 204. The bearing surfaces 252, 261 may be a dovetail configuration, as in the illustrated exemplary embodiment, to facilitate interlocking of the first section 204 to the second section 206. One or both of the bearing surfaces 252, 261 may have surface features, such as surface texturing, ridges, grooves, etc., to facilitate interlocking. Alternate ways of coupling the first and second sections may be used, such as inserting a snap ring into a groove in the second section that will also engage a groove in the first section.

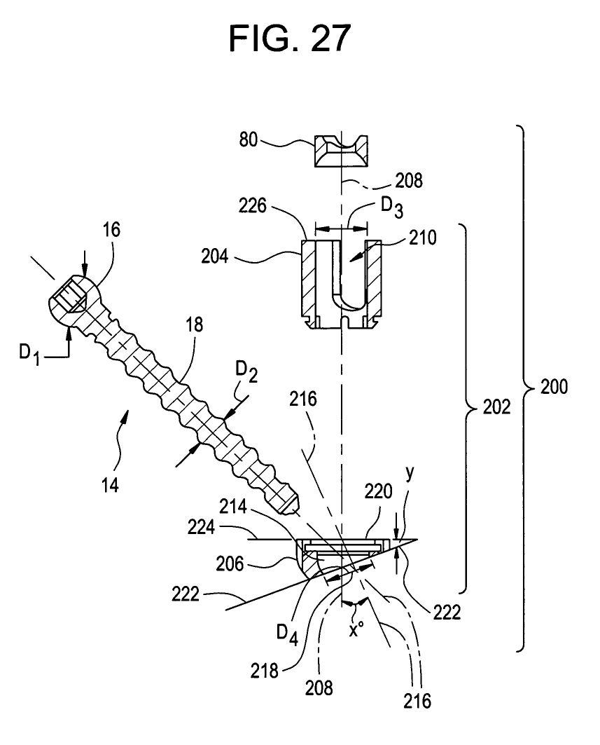

In certain exemplary embodiments, the bone anchor assemblies described herein may facilitate the incorporation of a bone anchor 14 having a larger diameter proximal head 16 and a larger diameter distal shaft 18. Referring to FIG. 27, for example, the diameter D₁ of the proximal head 16 of the bone anchor 14 and/or the diameter D₂ of the distal shaft 18 of the bone anchor 14, e.g., the major diameter of the distal shaft 18, may be greater than the diameter D₃ of the first bore 210 of the first section 204 of the proximal member 202. In addition, the diameter D₄ of the first opening 218 of the second section 206 of the proximal member 202 may be greater than the diameter D₃ of the first bore 210 of the first section 204 of the proximal member 202.

In the case of a bone anchor designed for use in the cervical spine, for example, the dimensions of the components of the bone anchor assembly may be:

TABLE 1
Large Anchor Diameter Cervical Bone Anchor Assembly

| Component | Diameter (mm) |
|---|---|
| Bone anchor head (D₁) | 6.0 mm |
| Bone anchor shaft (D₂) | 3.5 mm; 4.0 mm; 4.35 mm; |
| | 5.0 mm; 5.5 mm |
| First bore (D₃) of first | 5.0 mm |
| section | |
| First opening (D₄) of | 5.6 mm |
| second section | |

In the case of a bone anchor designed for use in the lumbar spine, for example, the dimensions of the components of the bone anchor assembly may be:

TABLE 2
Large Anchor Diameter Lumbar Bone Anchor Assembly

| Component | Diameter (mm) |
|---|---|
| Bone anchor head (D₁) | 10.0 mm |
| Bone anchor shaft (D₂) | 4.35 mm; 5.0 mm; 6.0 mm; |
| | 7.0 mm; 8.0 mm; 9.0 mm; |
| | 10.0 mm |
| First Bore (D₃) of first | 7.0 mm |
| section | |
| First opening (D₄) of | 9.3 mm |
| second section | |

In one exemplary method of manufacturing a bone anchor assembly, the bone anchor 14 may be inserted into the first opening 218 of the second section 206 prior to assembly of the second section 206 to the first section 204. In certain exemplary embodiments, the second section 206 may be movably connected to the first section 204 to facilitate movement, for example, rotation, of the second section 206 relative to the first section 204, as discussed above. In other exemplary embodiments, the second section 206 may be fixedly connected to the first section 204 to inhibit motion of the second section 206 relative to the first section 204. For example, the second section 206 may be fixed to the first section 204 by welding the sections together or via a press-fit. In certain exemplary embodiments, the angle Y between planes 222 and 224 may be 0°. In those exemplary embodiments where diameter D₂ of the distal shaft 18 of bone anchor 14 is larger than diameter D₄ of the first opening 218 of the second section 206 of proximal member 202, the first opening 218 of the second section 206 of the proximal member 202 may be threaded to facilitate the passage of distal shaft 18 of bone anchor 14.

While the bone anchor assemblies and methods of the present invention have been particularly shown and described with reference to the exemplary embodiments thereof, those of ordinary skill in the art will understand that various changes may be made in the form and details herein without departing from the spirit and scope of the present invention. Those of ordinary skill in the art will recognize or be able to ascertain many equivalents to the exemplary embodiments described specifically herein by using no more than routine experimentation. Such equivalents are intended to be encompassed by the scope of the present invention and the appended claims.

1. A bone anchor assembly comprising:
a bone anchor having a proximal head and a distal shaft configured to engage bone, the distal shaft having a shaft diameter and a longitudinal axis; and
a receiving member for receiving a spinal fixation element to be coupled to the bone anchor, the receiving member having
a first section having a first bore defining a first bore axis,
a recess in communication with the first bore, the recess being sized and shaped to receive a spinal fixation element, and
a second section having a second bore defining a second bore axis and being sized to receive at least a portion of the bone anchor, the second section being movably connected to the first section to facilitate relative motion of the first section and the second section.

2. The bone anchor assembly of claim 1, wherein the second bore axis intersects the first bore axis.

3. The bone anchor assembly of claim 2, wherein the second bore axis is oriented at angle of approximately 15° to approximately 70° relative to the first bore axis.

4. The bone anchor assembly of claim 1, wherein the first section defines a first plane, the second section defines a second plane, and the first and second plane intersect one another.

5. The bone anchor assembly of claim 1, wherein the second section is rotatably connected to the first section.

6. The bone anchor assembly of claim 5, wherein the second section is rotatable 360° about the first bore axis.

7. The bone anchor assembly of claim 5, wherein the second section is rotatable less than 360° about the first bore axis.

8. The bone anchor assembly of claim 1, further comprising a compression member positionable within the receiving member between the fixation element and the head of the bone anchor, the compression member having a first surface for engaging the spinal fixation element and an opposing second surface for engaging the head of the bone anchor.

9. The bone anchor assembly of claim 1, wherein the spinal fixation element is a spinal rod.

10. The bone anchor assembly of claim 1, wherein the head of the bone anchor includes a generally hemispherically shaped distal surface that engages a generally hemispherically shaped seat provided at the first opening of the second bore.

11. The bone anchor assembly of claim 1, wherein the bone anchor is adjustable relative to the receiving member.

12. The bone anchor assembly of claim 1, further comprising a closure mechanism engageable to the first end of the receiving member, the closure mechanism securing the spinal fixation element within the recess when engaged to the first section of the receiving member.

13. The bone anchor assembly of claim 12, wherein the closure mechanism locks the first section relative to the second section when engaged to the bone anchor assembly.

14. The bone anchor assembly of claim 12, wherein the closure mechanism engages an outer surface of the first end of the receiving member.

15. The bone anchor assembly of claim 12, wherein the closure mechanism seats within the first bore of the receiving member.

16. The bone anchor assembly of claim 1, wherein the first bore is coaxial to the second bore.

17. A bone anchor assembly comprising:
a bone anchor having a distal shaft configured to engage bone; and
a proximal member having
a first section, and
a second section coupled to at least a portion of the bone anchor, the second section being rotatably connected to the first section to facilitate relative rotation of the first section and the second section.

18. A bone anchor assembly comprising:
a bone anchor having a distal shaft configured to engage bone; and
a proximal member having
a first section having a first opening that defines a first plane, and
a second section coupled to at least a portion of the bone anchor, the second section having a second opening defining a second opening that defines a second plane that intersects the first plane, the second section being rotatably connected to the first section to facilitate relative rotation of the first section and the second section.

19. A bone anchor assembly comprising:
a bone anchor having a proximal head and a distal shaft configured to engage bone, the distal shaft having a shaft diameter; and
a receiving member for receiving a spinal fixation element to be coupled to the bone anchor, the receiving member having
a first section having a first bore having a proximal opening,
a recess in communication with the first bore, the recess being sized and shaped to receive a spinal fixation element, and
a second section having a second bore having a distal opening and being sized to receive at least a portion of the bone anchor, wherein a diameter of the distal opening of the second bore is greater than a diameter of the proximal opening of the first bore.

20. The bone anchor assembly of claim 19, wherein a diameter of the proximal head is greater than the diameter of the proximal opening of the first bore.

21. The bone anchor assembly of claim 19, wherein the shaft diameter is greater than the diameter of the proximal opening of the first bore.
